(19) United States
(12) Patent Application Publication
(10) Pub. No.: US 2015/0112105 A1
(43) Pub. Date: Apr. 23, 2015

(54) ASSESSMENT METHOD FOR HYDRATE INHIBITORS AND FORMULATION OF GAS HYDRATE INHIBITORS

(71) Applicant: Intevep, S.A., Caracas (VE)

(72) Inventors: Alfredo Viloria, Caracas (VE); Luis A. Castillo, Estado Guarico (VE); Jose A. García, Estado Miranda (VE); Rosa D. Nadales, Caracas (VE); Elluz V. Torín, Estado Lara (VE); María A. Carrasquero, Estado Carabobo (VE); María Llamedo, Caracas (VE)

(73) Assignee: Intevep, S.A., Caracas (VE)

(21) Appl. No.: 14/059,573

(22) Filed: Oct. 22, 2013

(51) Int. Cl. C07C 7/20; G01N 27/02; G01N 25/00; C07C 46/10; C09K 15/08

(52) U.S. Cl. CPC C07C 7/20; C07C 46/10; C09K 15/08; G01N 25/00; G01N 27/02
USPC 585/3; 568/324; 252/404; 73/53.01

(57) ABSTRACT

A gas hydrate inhibitor composition including an aqueous solution of anthraquinones. Methods of making and using the inhibitor are also provided, as well as a method of evaluating effectiveness of an inhibitor using THF.

### BACKGROUND OF THE INVENTION

The invention relates to a formulation and assessment method for a gas hydrate inhibitor application of gas hydrates in hydrocarbon production, transportation and treatment facilities.

In hydrocarbon production facilities such as off-shore platforms, pipelines, refineries, reactors and the like, under certain conditions natural gas hydrates can form which are ice-like crystalline solids formed of water and low molecular weight molecules trapped into the crystal lattice. This is particularly true when the hydrocarbon and water are exposed to temperatures below 20° C. combined with pressures above 1500 psia (10 atm).

These natural gas hydrates can cause flow problems leading to decreased productivity, can obstruct pipelines and other facilities, generate high costs for chemical treatments to remove them, and create risks to the safety of personnel as well as the installations during maintenance.

Hydrate inhibitors are known and can effectively be used to inhibit the formation of such natural hydrates. Known hydrate inhibitors include anti-aglomerants, kinetic hydrate inhibitors and thermodynamic hydrate inhibitors. Unfortunately, these hydrate inhibitors are expensive and are further undesirable from an environmental standpoint. In particular, thermodynamic inhibitors are very expensive because they require high dosage to be effective (up to 60% v/v).

The need therefore remains for a hydrate inhibitor which addresses both the high costs and environmental impact of known hydrate inhibitors.

It is therefore an object of this invention to provide a hydrate inhibitor which is relatively low in cost and friendlier from an environmental standpoint, and potentially more economic due to lower dosage needed in comparison to dosage needed with thermodynamic hydrate inhibitors.

### SUMMARY OF THE INVENTION

In accordance with the present invention, the foregoing object and others have been attained.

According to the invention, a gas hydrate inhibitor composition is provided which comprises an aqueous solution of anthraquinones.

In further accordance with the invention, a method is provided for evaluating a hydrate inhibitor in a laboratory setting. This method is based on monitoring temperature and electrical conductivity of an aqueous solution of tetrahydrofuran (THF). THF is known to form simple (single guest), stoichiometric, cubic structure-II clathrates hydrates of formula THF.17H₂O (equivalent to complete large hexakaidecahedral (51264) cage occupancy) which are stable at atmospheric pressure and temperature below 278K. The THF aqueous solution serves as a reference (blank) when it is studied without the presence of hydrate inhibitors. The THF aqueous solution is known for its ability to form hydrates of similar structures to gas hydrates, and thus is an excellent blank for evaluating effectiveness of gas hydrate inhibitors.

Still further according to the invention, a method is provided for producing the composition of the present invention, which method comprises the steps of obtaining raw material selected from a plant source selected from the group consisting of the leguminosae family, rhamnaceae family, the asphodelaceae family, the liliaceae family, the polygonaceae family and combinations thereof; pressing the raw material to produce a solid pressed raw material; and extracting anthraquinone from the solid pressed raw material. Further details of the extraction are discussed herein.

### BRIEF DESCRIPTION OF THE DRAWINGS

A detailed description of preferred embodiment of the invention follows, with reference to the attached drawings, wherein:

FIG. 1 shows the chemical composition of a basic anthraquinone;

FIG. 2 shows aloe emodin, a natural anthraquinone;

FIG. 3 shows barbaloin, an anthraquinone;

FIG. 4 shows emodin, an anthraquinone;

FIG. 5 shows an analysis of a natural anthraquinone according to the invention;

FIG. 6 shows a further analysis of an anthraquinone according to the invention;

FIG. 7 shows conductivity and temperature changes in a THF-aqueous solution system which is subject to hydrate formation;

FIG. 8 shows conductivity and temperature of a system which is protected by a typical conventional inhibitor;

FIG. 9 shows conductivity and temperature of a system which is protected with an anthraquinone inhibitor in accordance with the present invention; and

FIG. 10 schematically illustrates a method for preparing a composition in accordance with the present invention.

### DETAILED DESCRIPTION

The invention relates to a formulation based on anthraquinones as a hydrate inhibitor, and an assessment method for hydrate inhibitors. The hydrate inhibitors are applicable in scenarios where hydrates represent flow assurance problems, for example transportation or other handling of a fluid containing water and a hydrocarbon, when exposed to temperatures below 20° C. combined with pressures above 1500 psia (10 atm). Typically, these conditions are observed in underwater environments such as off-shore hydrocarbon production facilities, as one example.

In accordance with the invention, the formation of such gas hydrates is effectively inhibited by a composition comprising an aqueous solution of anthraquinones. As will be further discussed below, this composition is environmentally friendly, relatively low in cost due to lower dosage, and equally as effective as conventional high cost and highly toxic inhibitors of gas hydrate formation.

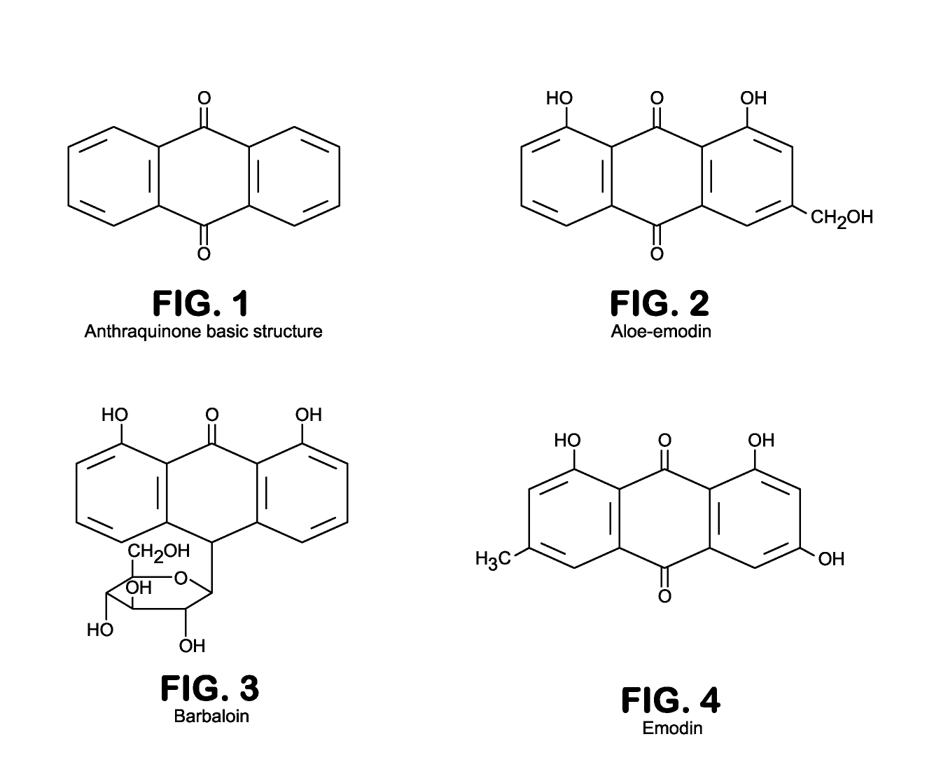

An anthraquinone is a colorless crystalline quinone, and can be chemically synthesized, for example by reacting benzene with phthalic anhydride. The basic structure of an anthraquinone is as shown in FIG. 1.

In accordance with the present invention, natural sources of anthraquinones have been identified, and the anthraquinones obtained from such natural sources are found according to the invention to be highly effective in inhibiting the formation of gas hydrates.

The anthraquinones according to the present invention can advantageously be obtained from plant sources such as the leguminosae family, the rhamnaceae family, the asphodelaceae family, the liliaceae family, the polygonaceae family and combinations thereof. Specific examples of plant sources of the anthraquinones for use in accordance with the present invention include Cassia siamea Britt.; C. occidentalis Linn., C. Fistula Linn., C. tora Linn., C. Surattensis burm., F., and C. garrettiana Craib, Aloe vera and Aloe barbadensis. All plant anthraquinones can be obtained by several methods, including via organic and inorganic solvent and supercritical extraction.

The anthraquinones for use in the present invention are advantageously formulated into a composition which then can be added to various different water and hydrocarbon mixtures and flows to inhibit the formation of gas hydrates. The composition in accordance with the present invention preferably includes an aqueous solution of anthraquinone.

The aqueous solution of anthraquinone preferably contains anthraquinone at a concentration of between 5 and 50% v/v.

The composition in accordance with the present invention is a Newtonian liquid in form, and therefore can easily be handled and introduced into hydrocarbon facilities as needed in order to inhibit the formation of gas hydrates.

As specified above, natural sources of anthraquinones have been found in accordance with the present invention, and these natural sources provide an excellent source of anthraquinones in accordance with the present invention.

In this regard, FIG. 2 illustrates a specific embodiment of a natural anthraquinone in accordance with the present invention, specifically aloe emodin.

FIG. 3 shows a further specific natural anthraquinone found to be useful in accordance with the present invention, specifically barbaloin (C₂₁H₂₂O₉).

FIG. 4 shows a further natural anthraquinone in accordance with the present invention, specifically emodin.

The anthraquinones are polyhydroxylated aromatic compounds, with various degrees of methylation. They can be found in a free state, or in glycosidic combinations, that is, linked together by molecules of sugar. For purposes of the present invention, the anthraquinones in free state and/or in glycosidic combinations can be used in order to formulate the hydrate inhibitor mentioned above.

The bark and roots of various plants typically contain the natural anthraquinones which are desired for use in accordance with the present invention, and various extraction techniques as will be discussed below, and can generally be focused on the bark and root of the plants. Typical examples of plants which are ideal sources include polygonaceae, rhamnaceas, legumes and liliaceae, among others.

In accordance with the present invention, the hydrate inhibiting aqueous solution of anthraquinone can be added directly to hydrocarbon treatment facilities, for example to any suitable flow of hydrocarbon and water mixtures, which is exposed to temperatures where gas hydrates would typically form, namely temperatures below 20° C. combined with pressures above 1500 psia (10 atm). The composition of the present invention alters the hydrate formation temperature such that the minimum temperatures to which the hydrocarbon and water flows are to be exposed will not cause the formation of gas hydrates at the same pressure. This will help to avoid all problems raised by formation of natural gas hydrates while minimizing the environmental impact from the treatment. This leads to reduction of chemical treatment processes, especially in off-shore operations, where colder conditions are encountered and disposal of environmentally hazardous materials is even more problematic. Further, environmentally hazardous synthesized inhibitors can be replaced with an equally effective inhibitor made from raw materials of the plant kingdom which are typically available in tropical areas local to the hydrocarbon facilities.

The gas hydrate inhibitor composition in accordance with the present invention can effectively be used by adding the composition to the hydrocarbon and water mixture under typical conditions, which may include the following: temperatures below 20° C. combined with pressures above 1500 psia (10 atm). These conditions are commonly observed in off-shore operations.

The amount required to achieve the effectiveness of the hydrate inhibitor is preferably between 5 and 30% v/v. In comparison, conventional thermodynamic hydrate inhibitors are applied in concentration up to 60% v/v.

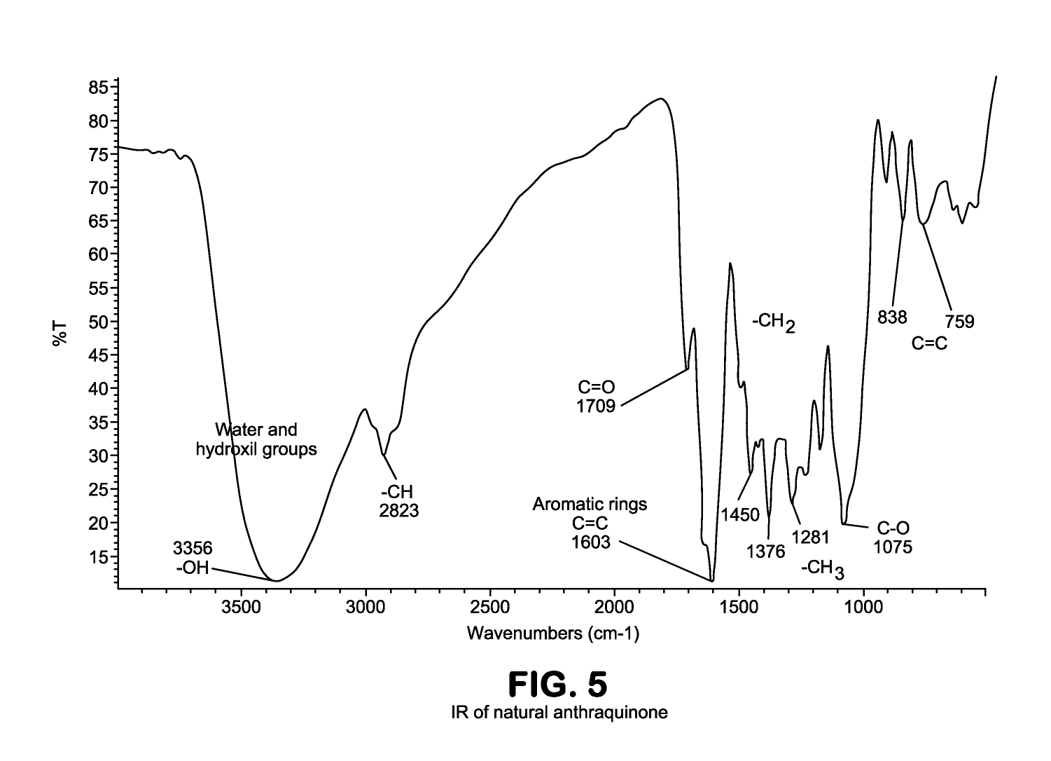

A natural anthraquinone in accordance with the present invention, namely barbaloin, was analyzed for various different chemical constituencies, and the results are shown in FIG. 5. The various different structures in barbarloin are illustrated by the peaks in FIG. 5 which indicate that the structure is a natural anthraquinone.

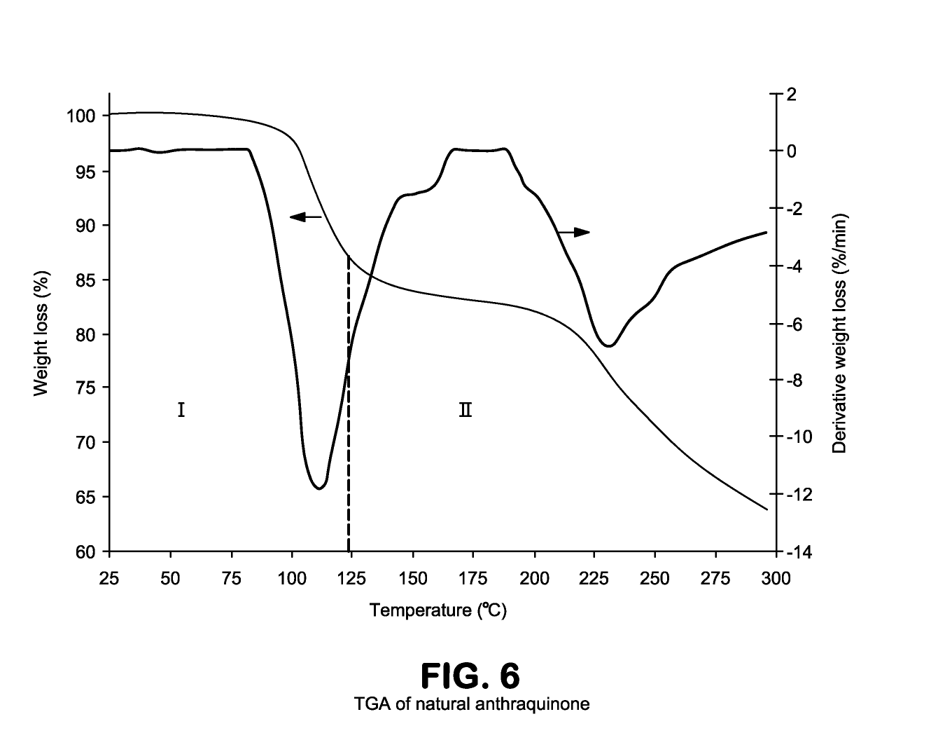

FIG. 6 illustrates weight loss and derivative weight loss at various temperatures for the natural anthraquinones in accordance with the present invention. This figure shows the thermal degradation temperature of a typical anthraquinone which could be used as an active compound of a hydrate inhibitor according to the invention. This temperature of degradation is higher than those required for hydrate formation. Thus, it will display a good performance at lower temperatures.

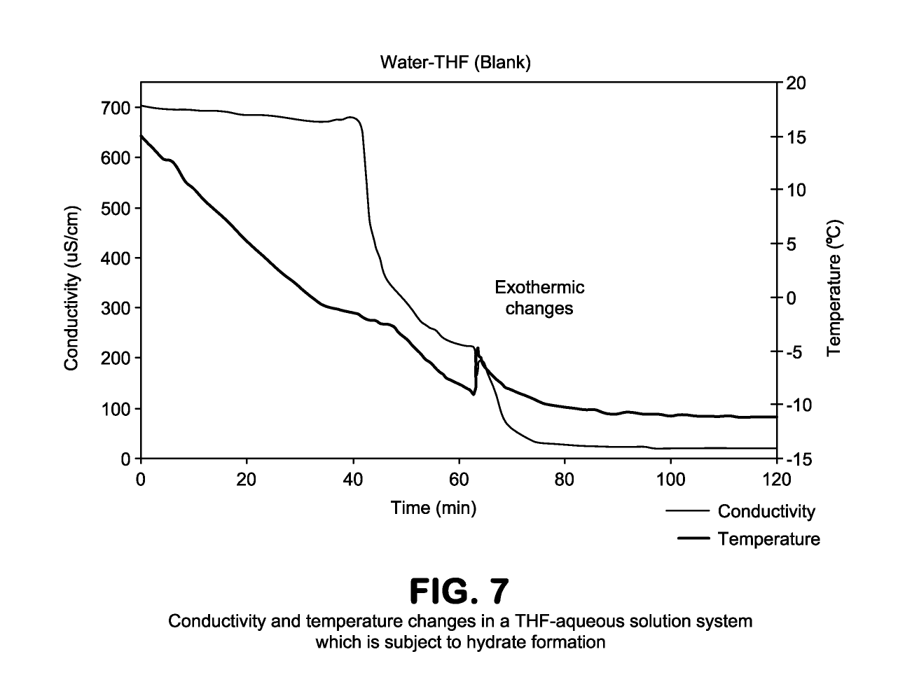

For purposes of the present invention, the effectiveness of any hydrate inhibitor can be determined by measuring the conductivity and temperature of a THF aqueous solution system over time. FIG. 7 shows a plot of conductivity and temperature over time for a THF aqueous solution system without any hydrate inhibitor (blank). The spike at approximately 60 minutes on this figure shows a decrease in conductivity which is typical at a point of formation of hydrates. The gradual decrease in conductivity likewise exhibits formation of hydrates. This conductivity decrease matched with exothermic changes of temperature are typical of hydrate formation reactions.

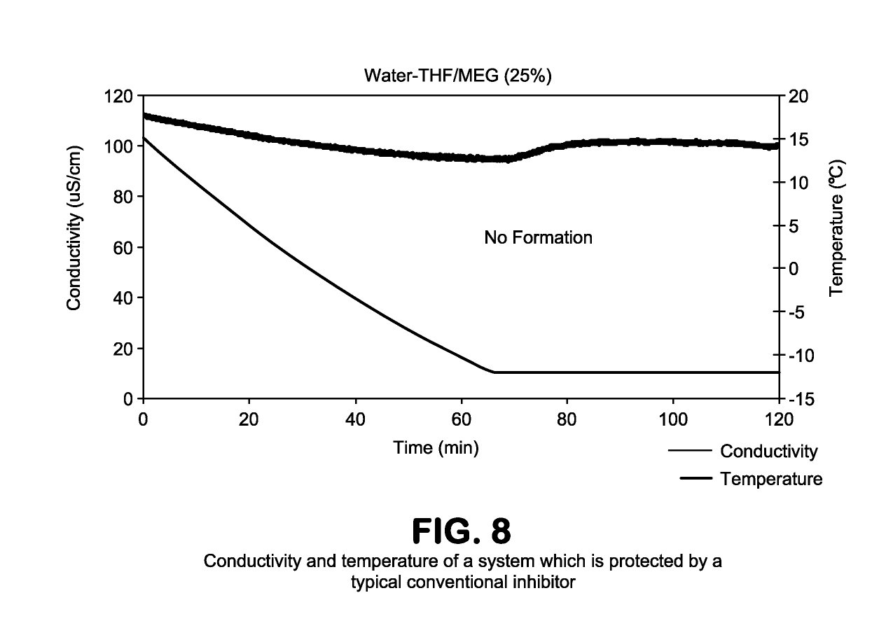

FIG. 8 shows the same conductivity and temperature for a THF aqueous solution system after the system has been treated with a conventional hydrate inhibitor (monoethylene glycol, MEG) added in a concentration of 25% v/v. As shown in FIG. 8, the temperature decrease over time is a smooth decline and then steadily remains without any spikes indicative of an exothermic change. Further, the conductivity of this system remains substantially constant. These curve profiles indicate that hydrate formation has been inhibited due to the presence of MEG.

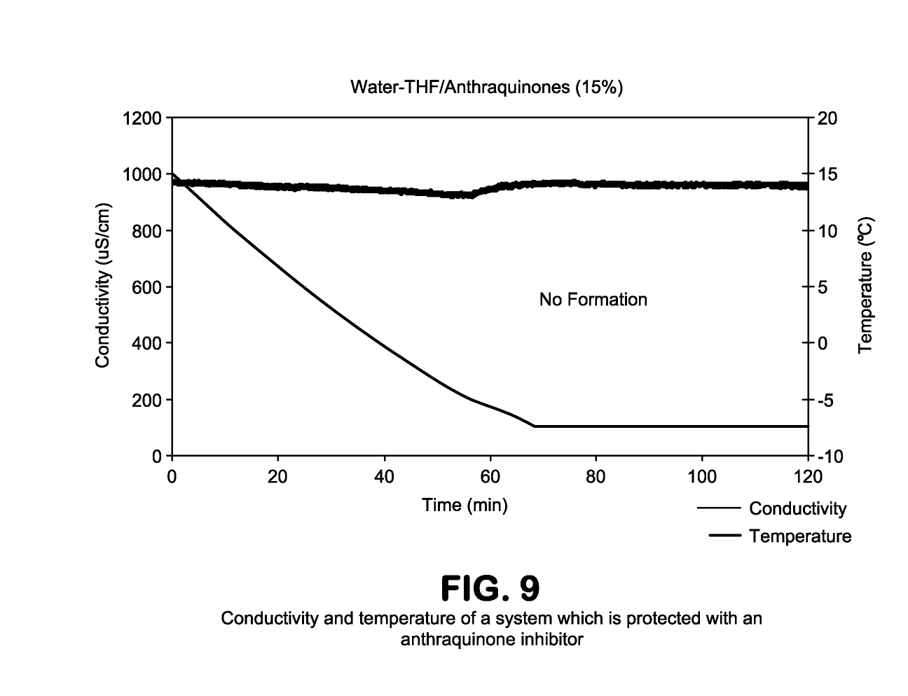

FIG. 9 shows a similar conductivity and temperature analysis for a THF aqueous solution system treated 15% v/v of aqueous solution of anthraquinones obtained from Aloe vera sap, in accordance with the present invention. FIG. 9 shows a likewise steady decrease in temperature over time with no exothermic spike, and substantially constant conductivity, and in fact the curve of FIG. 9 is nearly identical to that of FIG. 8. Thus, the composition in accordance with the present invention is equally as effective as known hydrate inhibitors, while the composition of the present invention is environmentally friendly and thereby advantageous over the known compositions. Additionally, the hydrate inhibitor based on anthraquinones in accordance with the present invention provides effectiveness at lower concentrations than conventional inhibitors (MEG, in this example).

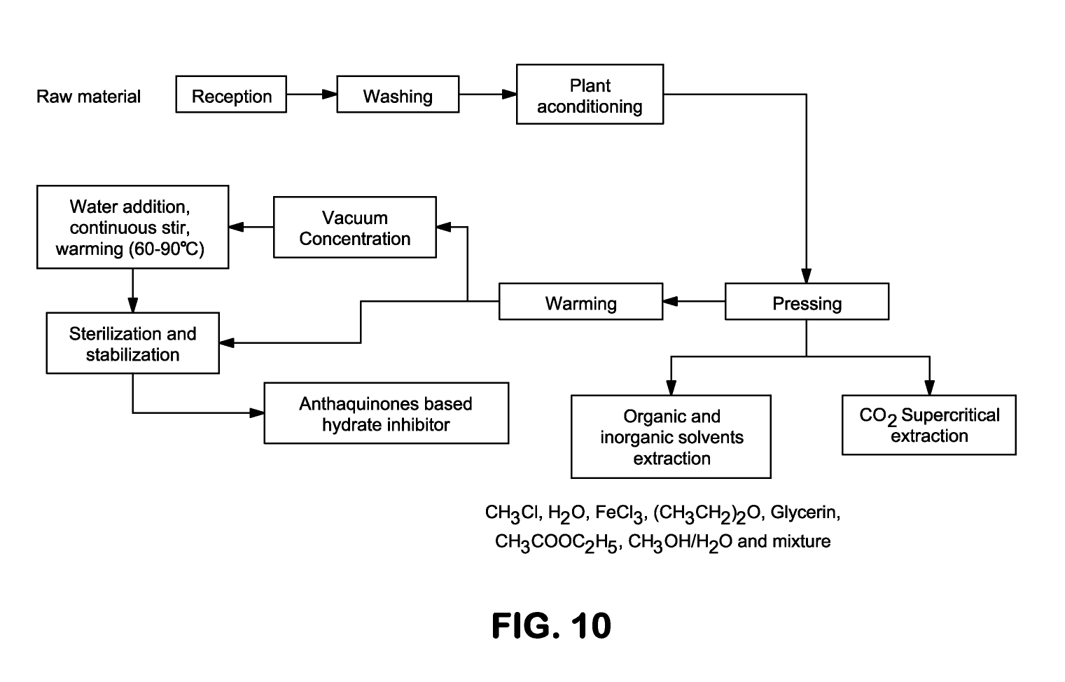

FIG. 10 schematically illustrates various alternatives for use in preparing anthraquinones to be incorporated into gas hydrate inhibitors according to the invention.

As shown in FIG. 10, the process can start with raw materials obtained from any of the various plant sources as discussed above. These plant sources, typically including the bark and roots of the plant, are preferably washed and conditioned with brushes for removing impurities, and then they are immersed in disinfectant solution of dilute sodium hipochloride, and then fed to a pressing step. In the pressing step, liquids are pressed from the solids, and both of these components can be treated in accordance with the present invention to obtain the desired anthraquinone materials.

As shown in FIG. 10, liquid from the pressed raw material can be fed to a warming step, and then alternatively either fed directly to sterilization and stabilization, or can first be fed to a vacuum concentration step followed by addition of water under continuous stirring and warming to temperature of between 60° C. and 90° C. Following vacuum concentration and mixing with water, this system can then be sterilized and stabilized to produce a desired natural anthraquinone in accordance with the present invention.

The solid raw materials from the pressing step can be passed to either an organic or an inorganic solvent extraction, or to CO₂ supercritical extraction, or both. The resulting materials are anthraquinones as well as CH₃Cl, H₂O, FeCl₃, (CH₃ CH₂)₂, Glucerin, CH₃COOC₂H₅, CH₃OH/H₂O.

Depending on the plant used as source, various anthraquinones can be extracted. For example, if the plant source is Aloe vera, then aloin will be extracted.

The composition of the present invention compares favorably to conventional hydrate inhibitors in a number of ways. Table 1 below sets forth typical properties of a conventional hydrate inhibitor. In contrast, Table 2 sets forth properties of a natural anthraquinone based hydrate inhibitor in accordance with the present invention.

TABLE 1
Conventional Hydrate Inhibitors

| — | Methanol | Ethylene glycol |
|---|---|---|
| Volatility | High | Low |
| Losses to the gas phase | 16 kg/10⁶Sm³ gas | 0.3kg/10⁶Sm³ gas |
| (by % inhibitor in water | (4° C. - 70 bar) | (4° C. - 70 bar) |
| phase) | | |
| Losses to the condensate | 0.5% weight | 0.03% of water phase |
| phase | | |
| Dosage | 10-50% weight | 10-50% weight |
| Final provision | To ambient | Regenerable |
| Environmental impact | Moderate | Moderate |
| Toxicity | High | Moderate |
| Cost | 300 $/Ton | 900 $/Ton |

TABLE 2
Non Conventional Anthraquinones based
hydrate inhibitor characterization

| Property | Value |
|---|---|
| Chemical composition | Hydrocarbon structure with |
| | presence of hydroxyl, carbonyl |
| | and methyl groups |
| pH | 3.6 |
| pKa | 5.2 |
| Fluid type | Newtonian |
| Volatility | Low |
| Estability | Chemical and thermal |
| In line dosification | 5-30% weight |
| Molecular Weight | >10 kDa |
| Solubility | Polar compounds |
| Environmental Impact | Environmentally friendly |
| Toxicity | Green chemistry |

Active principle
(Hydrate inhibitor)
Rhein anthraquinone
Chrysophanol
Aloe-emodin
Emodin

| Element | Range (%) |
|---|---|
| Oxygen | 40-60 |
| Carbon | 20-40 |
| Hydrogen | 2-15 |
| Nitrogen | <1 |
| Sulfur | <1 |
| Calcium | <1 |
| Magnesiu, | <1 |
| Other | 10-40 |

One or more embodiment of the present invention has been described herein. Nevertheless, it will be understood that various modifications may be made without departing from the spirit and scope of the invention. Accordingly, the embodiments are within the scopes of the invention as defined by the following claims.

1. A gas hydrate inhibitor composition, comprising an aqueous solution of anthraquinone.

2. The composition of claim 1, wherein the anthraquinone is an extract from a plant source selected from the group consisting of the leguminosae family, the rhamnaceae family, the asphodelaceae family, the liliaceae family, the polygonaceae family and combinations thereof.

3. The composition of claim 1, wherein the aqueous solution contains anthraquinones at a concentration between 5 and 50% v/v.

4. The composition of claim 1, wherein the anthraquinone has the following elemental composition:

| | |
|---|---|
| Oxygen (%) | 40-60 |
| Carbon | 20-40 |
| Hydrogen | 2-15 |
| Nitrogen | <1 |
| Sulfur | <1 |
| Calcium | <1 |
| Magnesium | <1 |
| Other | 10-40 |

5. A method for assessment of gas hydrate inhibitors, comprising monitoring conductivity and temperature of a system where THF hydrates can be formed.

6. The method of claim 5, wherein the tetrahydrofuran (THF) is a host molecule that lets hydrate formation proceed in presence of water.

7. A method for inhibiting formation of hydrates in a hydrocarbon flow, comprising adding to the flow a gas hydrate inhibitor composition comprising an aqueous solution of anthraquinone.

8. The method of claim 7, wherein the hydrate inhibitor composition is added to the hydrocarbon flow at a concentration between 5 and 30% v/v, and at conditions for hydrate formation.

9. A method for making a composition useful for inhibiting hydrate formations, comprising steps of:
obtaining raw material selected from a plant source selected from the group consisting of the leguminosae family, the rhamnaceae family, the asphodelaceae family, the liliaceae family, the polygonaceae family and combinations thereof
pressing the raw material to produce a solid pressed raw material; and
extracting anthraquinone from the solid pressed raw material.

10. The method of claim 9, wherein the extracting step includes CO₂ supercritical extraction.

11. The method of claim 9, wherein the extracting step includes a solvent extraction.

12. The method of claim 9, wherein the pressing step also produces a liquid fraction, and further comprising the steps of sterilizing and stabilizing the liquid fraction to obtain anthraquinone.

13. The method of claim 12, further comprising warming the liquid fraction to a temperature between 60 and 90° C., and adding water to the liquid faction, at concentrations between 5 and 50% of anthraquinones in water, before the sterilizing and stabilizing steps.
